(19) United States
(12) Patent Application Publication
(10) Pub. No.: US 2025/0345604 A1
(43) Pub. Date: Nov. 13, 2025

(54) INTRAVAGINAL ELECTRICAL STIMULATION DEVICE

(71) Applicant: Cornell University, Ithaca, NY (US)

(72) Inventor: Tamatha Fenster, Ithaca, NY (US)

(21) Appl. No.: 19/201,382

(22) Filed: May 7, 2025

Related U.S. Application Data
(60) Provisional application No. 63/644,969, filed on May 9, 2024.
(60) Provisional application No. 63/644,896, filed on May 9, 2024.

(51) Int. Cl. A61N 1/36; A61N 1/05

(52) U.S. Cl. CPC A61N 1/36007; A61N 1/0524; A61N 1/0551

(57) ABSTRACT

An intravaginal electrical stimulation device includes a vaginal insert and at least one electrically conductive electrode coupled to the vaginal insert. The vaginal insert is configured to be inserted into a vaginal canal of a user. With the vaginal insert inserted into the vaginal canal, at least a portion of the at least one electrically conductive electrode is arranged to contact a vaginal wall of the vaginal canal to electrically stimulate at least one pelvic nerve through the vaginal wall so that discomfort or pain associated with a pelvic condition or a procedure is alleviated.

### PRIORITY CLAIM

This application claims priority to U.S. Provisional Application No. 63/644,969 filed 9 May 2024 and U.S. Provisional Application No. 63/644,896 filed 9 May 2024, each of which are expressly incorporated by reference herein.

### BACKGROUND

Many women experience pelvic pain, such as pelvic pain from primary or secondary dysmenorrhea. Dysmenorrhea is the medical term for painful menstrual periods. Primary dysmenorrhea refers to recurrent pain with no identifiable cause. Secondary dysmenorrhea results from conditions such as endometriosis. These pelvic pain conditions significantly affect the quality of life of many individuals; however, existing treatment options are limited.

Existing treatment options include pharmacological treatments, such as nonsteroidal anti-inflammatory drugs (NSAIDs) or stronger pharmacological pain relivers (e.g., opioids), hormonal therapies (e.g., birth control), and transcutaneous electrical nerve stimulation (TENS) therapy. For secondary dysmenorrhea, surgical interventions may be used, such as laparoscopy, laparotomy, or hysterectomy. Many of these solutions are drug based and/or may not be effective at relieving pain and discomfort. Accordingly, there is a need to develop a device that alleviates acute or chronic pelvic pain, such as pain from primary or secondary dysmenorrhea.

### SUMMARY

According to the present disclosure, an intravaginal electrical stimulation device is described. The intravaginal electrical stimulation device is configured to be inserted into a vaginal canal of a user to electrically stimulate at least one pelvic nerve through a vaginal wall of the vaginal canal so that discomfort or pain associated with a pelvic condition is alleviated.

The intravaginal electrical stimulation device includes a vaginal insert that is inserted into the vaginal canal and at least one electrically conductive electrode coupled to the vaginal insert. With the vaginal insert positioned in the vaginal canal, at least a portion of the at least one electrically conductive electrode is arranged to contact the vaginal wall of the vaginal canal to electrically stimulate the at least one pelvic nerve through the vaginal wall.

The vaginal insert may be an insert, a menstrual cup, a menstrual disc, or a menstrual plug. In some embodiments, the vaginal insert is configured to hold, collect, or absorb fluid, such as menstrual fluid. In some embodiments, the vaginal insert is not configured to hold, collect, or absorb fluid.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 is a side diagrammatic view of an exemplary intravaginal electrical stimulation device positioned in a vaginal canal of a user;

FIG. 2 is a perspective view of the intravaginal electrical stimulation device of FIG. 1 showing a menstrual collector that defines a fluid-collection reservoir to hold fluid therein and at least one electrically conductive electrode coupled to the menstrual collector that is configured to electrically stimulate at least one pelvic nerve through a vaginal wall while the intravaginal electrical stimulation device is positioned in the vaginal canal of the user;

FIG. 3 is a perspective view of the intravaginal electrical stimulation device of FIG. 2 loaded in a deployment device to aid the user in inserting the intravaginal electrical stimulation device into the vaginal canal;

FIG. 4 is a back view of another embodiment of an intravaginal electrical stimulation device showing a menstrual collector and a plurality of electrically conductive electrodes coupled to a posterior side of the menstrual collector;

FIG. 5 is a side view of the intravaginal electrical stimulation device of FIG. 4 showing that the menstrual collector includes a body that the plurality of electrically conductive electrodes is coupled to and an angled rim coupled to the body that mimics a shape of the vaginal canal;

FIG. 6 is a front view of the intravaginal electrical stimulation device of FIG. 4 showing that the intravaginal electrical stimulation device further includes a cord coupled to the menstrual collector and a battery coupled to the cord to provide power to the plurality of electrically conductive electrodes;

FIG. 7 is a back view of another embodiment of an intravaginal electrical stimulation device showing a menstrual collector and a plurality of electrically conductive electrodes coupled to a posterior side of the menstrual collector and extending along a height of the menstrual collector;

FIG. 8 is a side view of the intravaginal electrical stimulation device of FIG. 7 showing that the menstrual collector includes a body that the plurality of electrically conductive electrodes is coupled to and an angled rim coupled to the body that mimics a shape of the vaginal canal;

FIG. 9 is a front view of the intravaginal electrical stimulation device of FIG. 7 showing that the intravaginal electrical stimulation device further includes a cord coupled to the menstrual collector and a battery coupled to the cord to provide power to the plurality of electrically conductive electrodes;

FIG. 10 is a back view of another embodiment of an intravaginal electrical stimulation device showing a menstrual collector and a plurality of electrically conductive electrodes coupled to an exterior wall of the menstrual collector;

FIG. 11 is a side view of the intravaginal electrical stimulation device of FIG. 10 showing that the menstrual collector includes a body that the plurality of electrically conductive electrodes is coupled to and an angled rim coupled to the body that mimics a shape of the vaginal canal;

FIG. 12 is a front view of the intravaginal electrical stimulation device of FIG. 10 showing that the plurality of electrically conductive electrodes is coupled to an interior wall of the menstrual collector;

FIG. 13 is a back view of another embodiment of an intravaginal electrical stimulation device showing a menstrual collector and a plurality of electrically conductive electrodes coupled to the menstrual collector;

FIG. 14 is a side view of the intravaginal electrical stimulation device of FIG. 13 showing that the menstrual collector includes a body and an angled rim coupled to the body that mimics a shape of the vaginal canal, and further showing that the plurality of electrically conductive electrodes is coupled to the body and the angled rim;

FIG. 15 is a front view of the intravaginal electrical stimulation device of FIG. 13 showing that the plurality of electrically conductive electrodes is coupled to the angled rim in spaced apart relation to one another about the angled rim;

FIG. 16 is a side diagrammatic view of another embodiment of an intravaginal electrical stimulation device positioned in a vaginal canal of a user;

FIG. 17 is a perspective view of the intravaginal electrical stimulation device of FIG. 16 showing a menstrual collector that defines a fluid-collection reservoir to absorb fluid therein and at least one electrically conductive electrode coupled to the menstrual collector that is configured to electrically stimulate at least one pelvic nerve through a vaginal wall while the intravaginal electrical stimulation device is positioned in the vaginal canal of the user;

FIG. 18 is a perspective view of the intravaginal electrical stimulation device of FIG. 17 loaded in a deployment device to aid the user in inserting the intravaginal electrical stimulation device into the vaginal canal;

FIG. 19 is a side view of another embodiment of an intravaginal electrical stimulation device showing a Y-shaped vaginal insert and a plurality of electrically conductive electrodes coupled to the Y-shaped vaginal insert;

FIG. 19A is a side view of another embodiment of an intravaginal electrical stimulation device showing a vaginal insert and a plurality of electrically conductive electrodes coupled to the vaginal insert;

FIG. 19B is a side view of another embodiment of an intravaginal electrical stimulation device showing a ring-shaped vaginal insert and a plurality of electrically conductive electrodes coupled to the ring-shaped vaginal insert;

FIG. 20 is a side diagrammatic view of another embodiment of an intravaginal electrical stimulation device positioned in a vaginal canal of a user;

FIG. 21 is a perspective view of the intravaginal electrical stimulation device of FIG. 20 while the intravaginal electrical stimulation device is not inserted into the vaginal canal of the user, and further showing that the intravaginal electrical stimulation device includes a menstrual collector that defines a fluid-collection reservoir to hold fluid therein and at least one electrically conductive electrode coupled to the menstrual collector that is configured to electrically stimulate at least one pelvic nerve through a vaginal wall while the intravaginal electrical stimulation device is positioned in the vaginal canal of the user;

FIG. 22 is a perspective view of the intravaginal electrical stimulation device of FIG. 21 while the intravaginal electrical stimulation device is inserted into the vaginal canal of the user;

FIG. 23 is a perspective view of a deployment device to aid the user in inserting and/or removing the intravaginal electrical stimulation device into and/or from the vaginal canal;

FIG. 24 is a perspective view of the intravaginal electrical stimulation device of FIG. 21 including a removal cord; and

FIG. 25 is a block diagram of a control system of the intravaginal electrical stimulation device.

### DETAILED DESCRIPTION

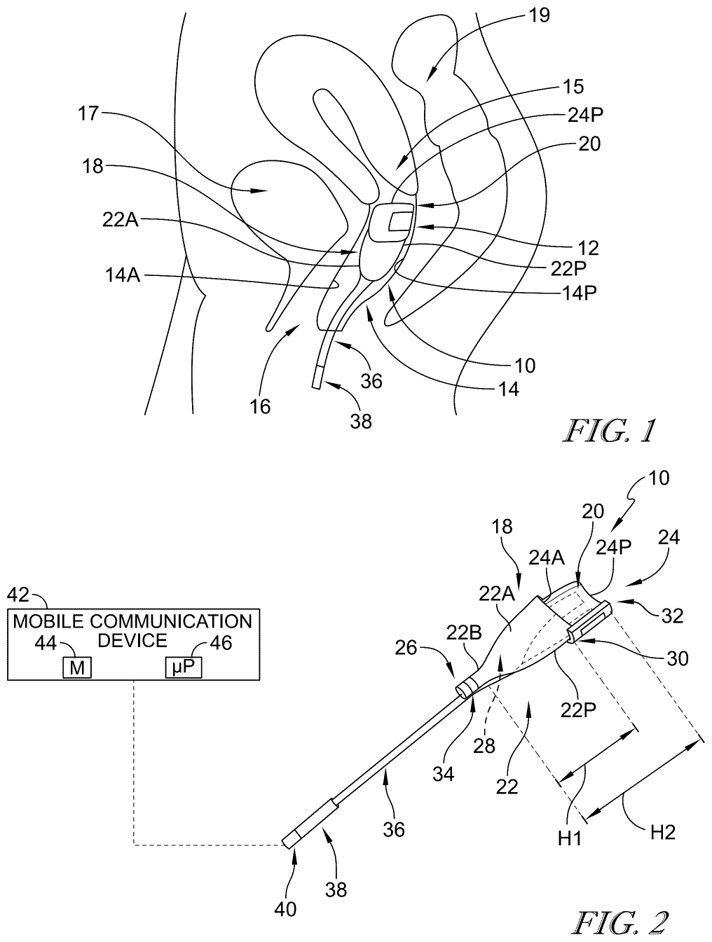

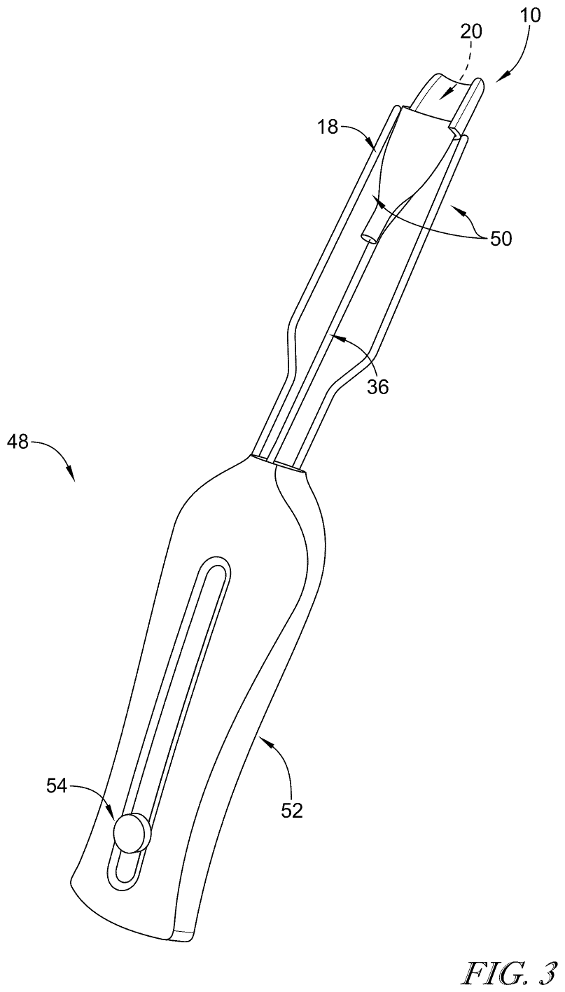

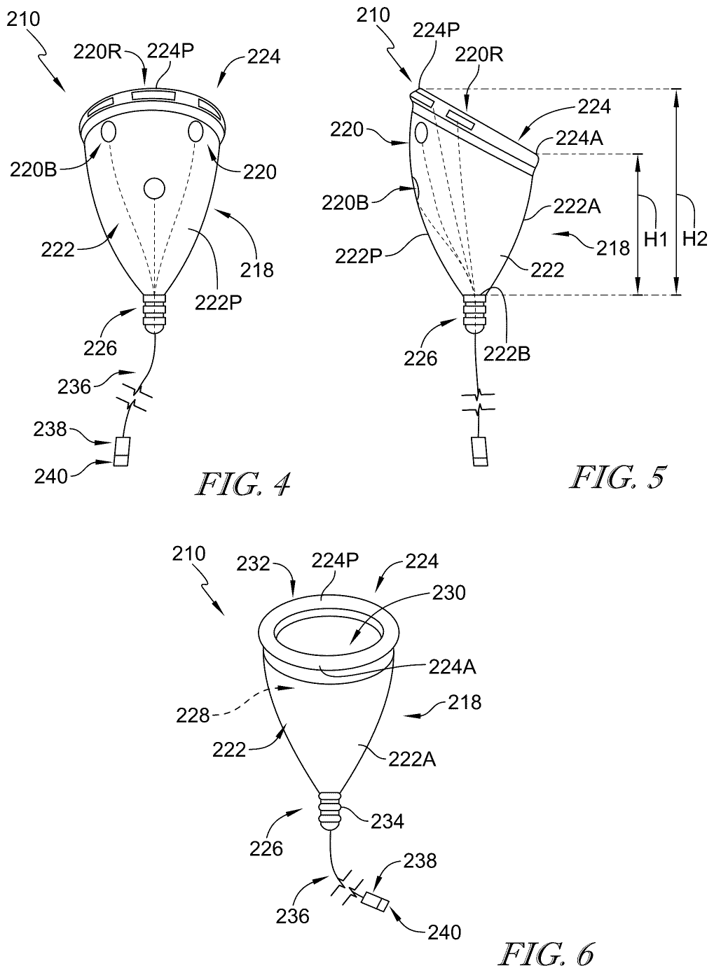

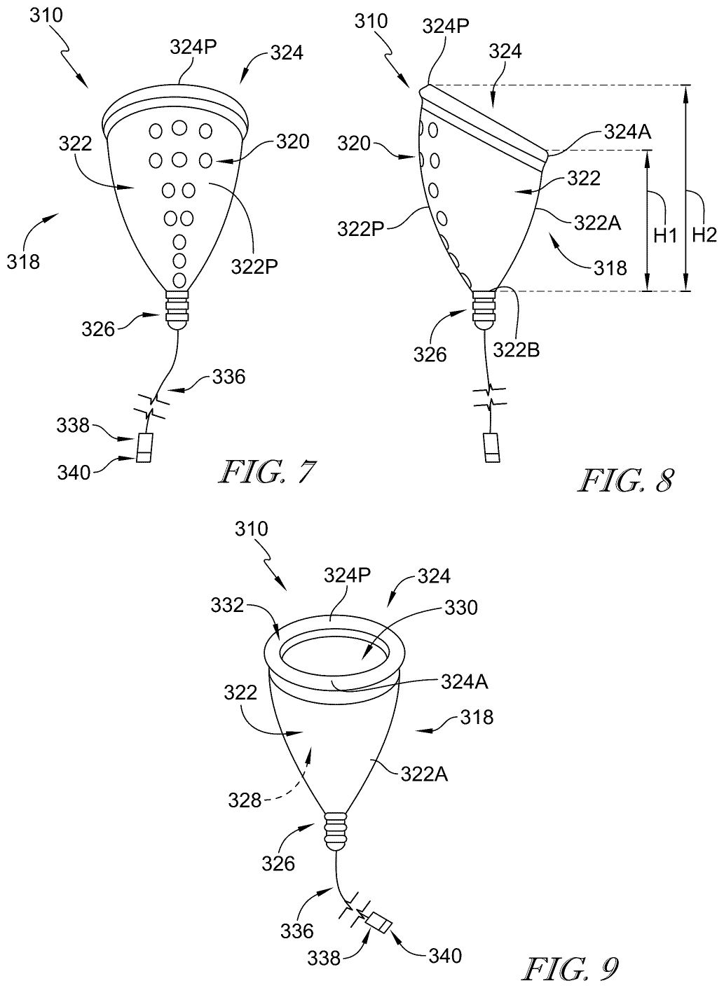

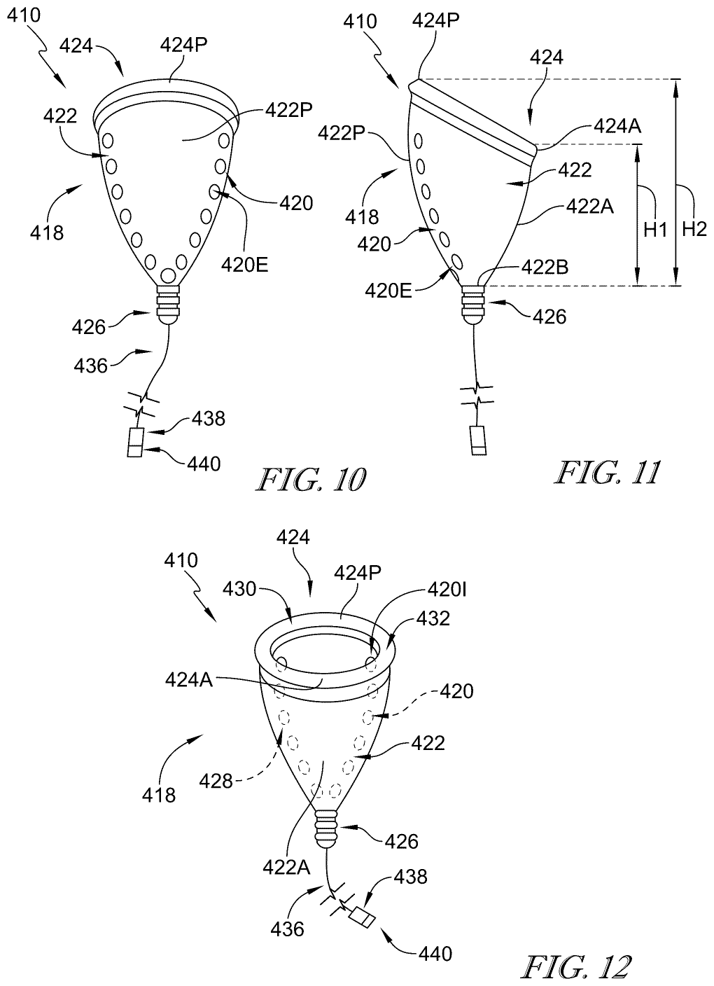

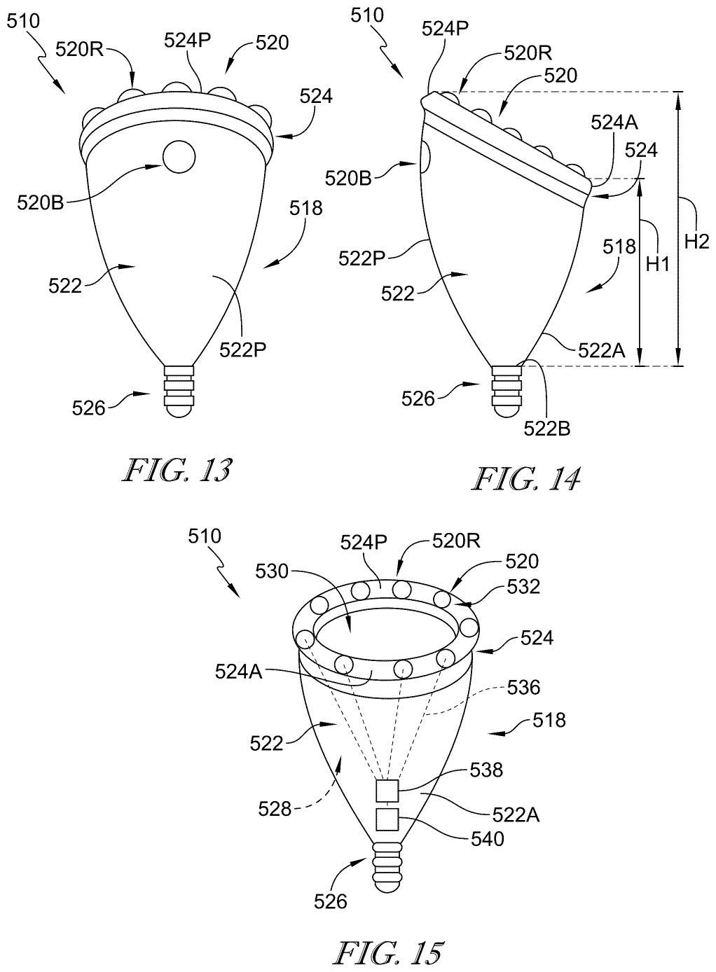

The present disclosure relates to an intravaginal electrical stimulation device 10 for electrically stimulating at least one pelvic nerve 12 through a vaginal wall 14 of a vaginal canal 16 so that discomfort or pain is alleviated, as suggested in FIG. 1. The discomfort or pain may be associated with a pelvic condition (e.g., physiologic pain). The pelvic condition may cause acute pelvic pain or chronic pelvic pain. The pelvic condition may be one or more of primary dysmenorrhea (e.g., menstrual cramps), secondary dysmenorrhea, endometriosis, adenomyosis, endometrial polyps, reproductive disease, pelvic inflammatory disease, congenital issues involving reproductive organs, uterine fibroids, polycystic ovary syndrome, interstitial cystitis, stress urinary incontinence, urge urinary incontinence, bladder urgency, fecal incontinence, among other pelvic conditions. The discomfort or pain may be associated with a procedure (e.g., procedural pain or operative pain). The procedure may be one or more of intrauterine device (IUD) insertion, hysteroscopy, endometrial biopsy, uterine fibroid embolization, fertility procedures, cervical dysplasia procedures, among other procedures. The at least one pelvic nerve 12 may be a hypogastric nerve, a pudendal nerve, or any other nerve within the vaginal canal 16.

The intravaginal electrical stimulation device 10 includes a menstrual collector 18 and at least one electrically conductive electrode 20, as shown in FIG. 2. The menstrual collector 18 is configured to be inserted into the vaginal canal 16 of the user. The at least one electrically conductive electrode 20 is coupled to the menstrual collector 18 such that the at least one electrically conductive electrode 20 is located within the vaginal canal 16 while the menstrual collector 18 is located within the vaginal canal 16.

In some embodiments, the menstrual collector 18 is a menstrual cup, as shown in FIG. 2, or a menstrual disc. The menstrual collector 18 includes a body 22 and a rim 24. The body 22 is shaped to define an interior region 28 that provides a fluid-collection reservoir 30 to hold fluid therein. The rim 24 is coupled to the body 22 and formed to include an opening 32 into the interior region 28.

In some embodiments, the menstrual collector 18 further includes a stem 26, as shown in FIG. 2. The stem 26 is coupled to the body 22 opposite the rim 24 at a bottom end 22B of the body 22. In some embodiments, the stem 26 is omitted.

The fluid-collection reservoir 30 of the body 22 may hold or collect fluid therein, such as menstrual fluid. In this way, the intravaginal electrical stimulation device 10 may be used by the user during menstruation. In some embodiments, the menstrual collector 18 includes a removable sponge (not shown) in the fluid-collection reservoir 30 that absorbs fluid within the fluid-collection reservoir 30. The intravaginal electrical stimulation device 10 may also be used outside of menstruation such that little to no fluid is held or collected in the fluid-collection reservoir 30.

In some embodiments, such as embodiments in which the menstrual collector 18 is a menstrual cup, the body 22 may be bell-shaped so that the body 22 seals against the vaginal wall 14 below a cervix 15 of the user while the intravaginal electrical stimulation device 10 is inserted in the vaginal canal 16. In some embodiments, such as embodiments in which the menstrual collector 18 is a menstrual disc, the body 22 may have a flat disc shape that rests below the cervix 15.

The body 22 defines an anterior side 22A (e.g., a front side) and a posterior side 22P (e.g., a back side), as shown in FIG. 2. The anterior side 22A faces, engages, or contacts an anterior wall 14A of the vaginal canal 16 located adjacent a bladder 17 of the user. The posterior side 22P faces, engages, or contacts a posterior wall 14P of the vaginal canal 16 located adjacent a rectum 19 of the user.

The rim 24 of the menstrual collector 18 defines an upper end of the body 22, as shown in FIG. 2. The opening 32 of the rim 24 is located adjacent the cervix 15 so as to receive fluid passing through the cervix 15 from a uterus of the user. As shown in FIG. 2, the rim 24 has a variable height from the bottom end 22B of the body 22 of the menstrual collector 18. An anterior portion 24A of the rim 24 at the anterior side 22A of the body 22 is located a first height H1 from the bottom end 22B of the body 22. A posterior portion 24P of the rim 24 at the posterior side 22P of the body 22 is located a second height H2 from the bottom end 22B of the body 22. In illustrative embodiments, the second height H2 is greater than the first height H1. The variable height enables the menstrual collector 18 to mimic the shape of the vaginal canal 16 (as the anterior wall 14A of the vaginal canal 16 is shorter than the posterior wall 14P of the vaginal canal 16). In this way, the posterior portion 24P of the rim 24, in some embodiments, contacts or engages a portion of the cervix 15 (e.g., the ectocervix) while the menstrual collector 18 is located in the vaginal canal 16.

The stem 26 extends away from the body 22, as shown in FIG. 2. In some embodiments, the stem 26 may be omitted, for example, if the menstrual collector 18 is a menstrual disc. In some embodiments, the stem 26 is formed to include one or more ridges 34. In some embodiments, the one or more ridges 34 may be omitted. The one or more ridges 34 may extend concentrically around the stem 26. The one or more ridges 34 help the user to grip the stem 26 during removal of the menstrual collector 18 from the vaginal canal 16.

The at least one electrically conductive electrode 20 is coupled to the menstrual collector 18 such that, with the menstrual collector 18 inserted into the vaginal canal 16, at least a portion of the at least one electrically conductive electrode 20 engages or contacts the vaginal wall 14 of the vaginal canal 16, as suggested in FIG. 1. To this end, the at least one electrically conductive electrode 20 is coupled to an exterior surface of the menstrual collector 18. The at least one electrically conductive electrode 20 is configured to electrically stimulate the at least one pelvic nerve 12 through the vaginal wall 14 so that discomfort or pain associated with the pelvic condition or the procedure is alleviated.

In some embodiments, the at least one electrically conductive electrode 20 is coupled to the posterior side 22P of the body 22 of the menstrual collector 18, as shown in FIG. 2. In such an embodiment, the at least one electrically conductive electrode 20 engages or contacts the posterior wall 14P of the vaginal canal 16.

In some embodiments, the at least one electrically conductive electrode 20 is coupled to the anterior side 22A of the body 22 of the menstrual collector 18. In such an embodiment, the at least one electrically conductive electrode 20 engages or contacts the anterior wall 14A of the vaginal canal 16.

In some embodiments, the at least one electrically conductive electrode 20 is coupled to the posterior portion 24P of the rim 24 of the menstrual collector 18. In such an embodiment, the at least one electrically conductive electrode 20 engages or contacts the cervix 15. In some embodiments, the at least one electrically conductive electrode 20 is coupled to the anterior portion 24A of the rim 24 of the menstrual collector 18.

In some embodiments, the at least one electrically conductive electrode 20 is coupled to one or more of the anterior side 22A of the body 22, the posterior side 22P of the body 22, the posterior portion 24P of the rim 24, and the anterior portion 24A of the rim 24. In some embodiments, the at least one electrically conductive electrode 20 includes more than one electrode. In such an embodiment, the at least one electrically conductive electrode 20 includes any number of electrodes, such as two (2) electrodes to thirty (30) electrodes, including any specific number comprised therein. The size, shape, and placement of the at least one electrically conductive electrode 20 may vary based on the at least one pelvic nerve 12 being targeted, the pelvic condition, the procedure, and/or the user. In some embodiments, the at least one electrically conductive electrode 20 comprises wire embedded, woven, and/or weaved into the menstrual collector 18. The at least one electrically conductive electrode 20 is illustratively malleable to allow for the intravaginal electrical stimulation device 10 to deform during insertion and/or removal of the intravaginal electrical stimulation device 10. In some embodiments, the at least one electrically conductive electrode 20 is radiopaque such that the at least one electrically conductive electrode 20 is visible on a sonogram, for example.

The at least one electrically conductive electrode 20 electrically stimulates the at least one pelvic nerve 12 to modulate nerve activity and muscle response. For example, the stimulation may cause sensory control and/or motor control. Motor control (i.e., muscle control) may influence uterine contractions and may decrease heavy menstrual bleeding. This modulation of nerve activity alleviates, reduces, or minimizes pain or discomfort stemming from the pelvic condition or the procedure without the side effects associated with traditional treatment options. The electrical stimulation may block the transmission of a pain signal by the at least one pelvic nerve 12.

For example, the intravaginal electrical stimulation device 10 may eliminate or reduce the need for pharmacological treatments or stronger pharmacological pain relivers. Long term use of NSAIDs may lead to side effects, such as stomach ulcers or gastric bleeding, and may increase the risk of or cause cardiovascular events, high blood pressure, kidney damage, etc. Likewise, opioid usage may lead to physical dependence or tolerance. In this way, the intravaginal electrical stimulation device 10 reduces the user's reliance on medication.

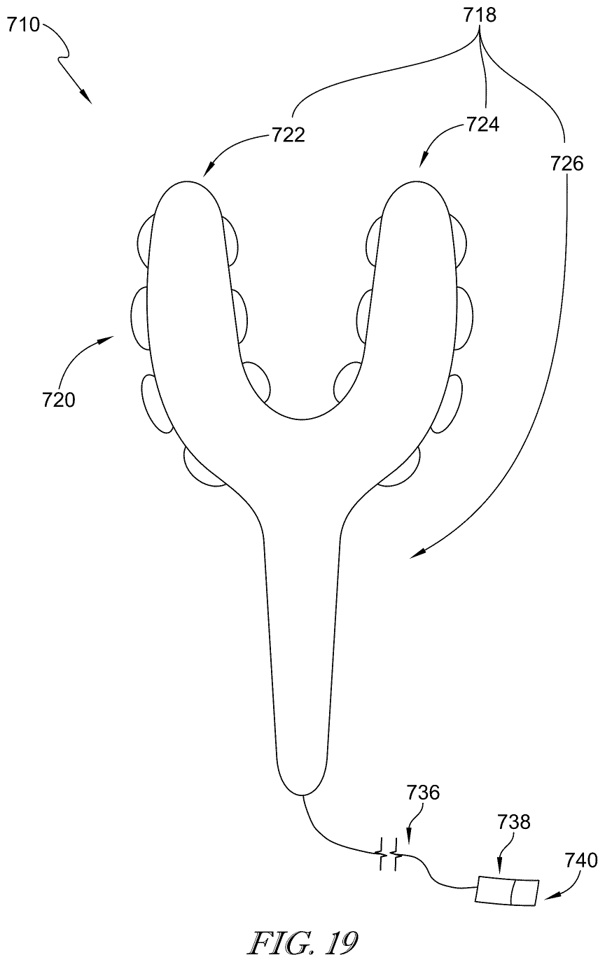

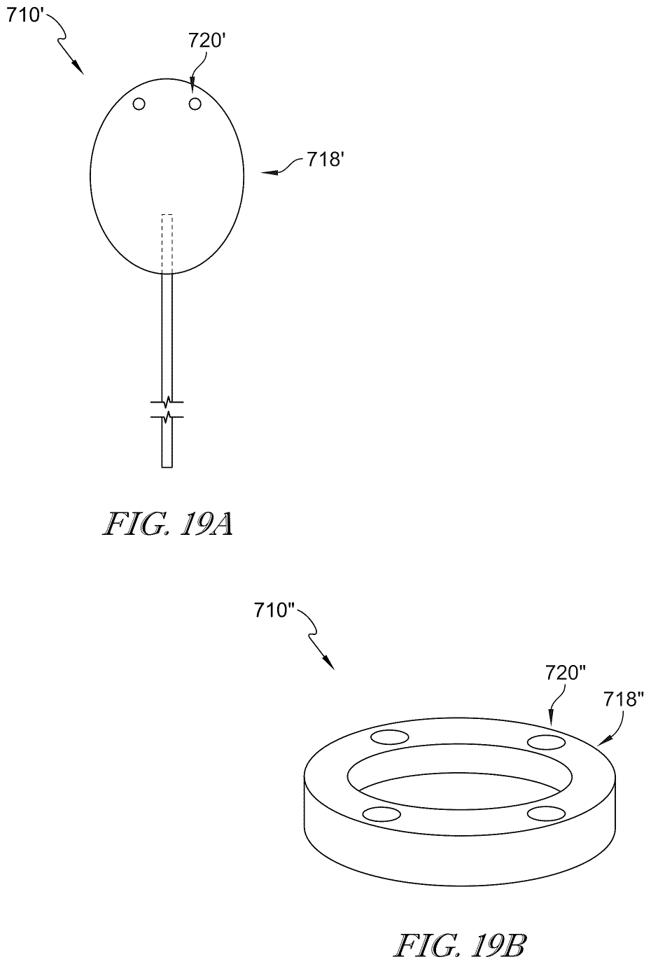

In some embodiments, the intravaginal electrical stimulation device 10 is used before, during, and/or after the procedure. For example, the intravaginal electrical stimulation device 10 may be used before IUD insertion, during IUD insertion, and/or after IUD insertion to alleviate, reduce, and/or minimize pain or discomfort stemming from the IU D insertion (see FIG. 19B).

Because the intravaginal electrical stimulation device 10 is positioned in the vaginal canal 16, the intravaginal electrical stimulation device 10 provides direct and localized treatment (as compared to TENS therapy) to the area of pain or discomfort, which improves efficacy and user comfort. In this way, the electrical stimulation is entirely intravaginal with no external electrodes located on a skin surface of the user. Further, the mimicking of the shape of the vaginal canal 16 by the menstrual collector 18 allows for more precise targeting of the at least one pelvic nerve 12. The at least one pelvic nerve 12 may be a pudendal nerve, which is often implicated in chronic pelvic pain. The at least one pelvic nerve 12 may also be a hypogastric nerve. The shape of the menstrual collector 18 also allows for engagement or contact between the posterior portion 24P of the rim 24 and the cervix 15, which optimizes the distribution of electrical signals for maximum therapeutic benefit.

In some embodiments, the intravaginal electrical stimulation device 10 further includes a cord 36 and/or a battery 38, as shown in FIGS. 1 and 2. The cord 36 is coupled to the stem 26 or the bottom end 22B of the body 22, the battery 38, and the at least one electrically conductive electrode 20.

The cord 36 is coupled to the menstrual collector 18 such that, with the menstrual collector 18 inserted into the vaginal canal 16, at least a portion of the cord 36 is located exterior to the vaginal canal 16, as shown in FIG. 1. The cord 36 extends between and interconnects the menstrual collector 18 and the battery 38 and the battery 38 and the at least one electrically conductive electrode 20. In some embodiments, a portion of the cord 36 that extends from the battery 38 to the at least one electrically conductive electrode 20 is located exterior to the menstrual collector 18. In some embodiments, a portion of the cord 36 that extends from the battery 38 to the at least one electrically conductive electrode 20 is located interior to the menstrual collector 18 (i.e., within the walls of the menstrual collector 18).

The battery 38 is connected to the cord 36 such that, with the menstrual collector 18 inserted into the vaginal canal 16, the battery 38 is located exterior to the vaginal canal 16, as shown in FIG. 1. The battery 38 may be a rechargeable battery or a replaceable battery. The cord 36 allows for transmission of power from the battery 38 to the at least one electrically conductive electrode 20 while the battery 38 is located exterior to the vaginal canal 16.

In some embodiments, the cord 36 and the battery 38 may be located entirely within the vaginal canal 16. In some embodiments, the at least one electrically conductive electrode 20 may be wirelessly powered such that the cord 36 is omitted and the battery 38 is located exterior to the vaginal canal 16 or within the vaginal canal 16. For example, the at least one electrically conductive electrode 20 may be powered through radio frequency waves, a thermoelectric generator (TEG), a piezoelectric generator, passive powering, or any other known wireless power transfer method.

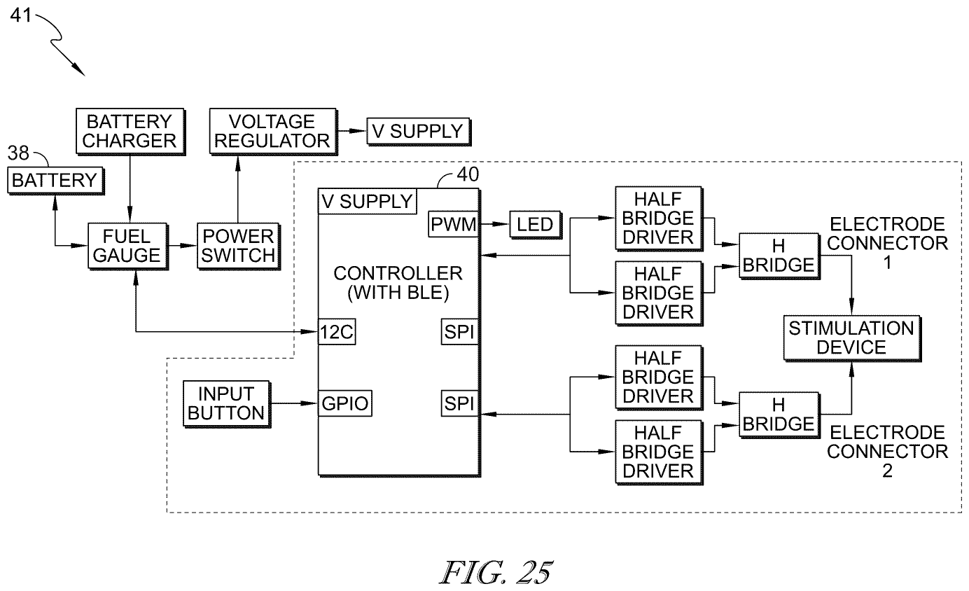

In some embodiments, the intravaginal electrical stimulation device 10 further includes a controller 40, as shown in FIG. 2. The controller 40 may form part of a control system 41, as shown in FIG. 25. The controller 40 is in communication with the at least one electrically conductive electrode 20 to send electrical signals thereto. The controller 40 selectively controls the at least one electrically conductive electrode 20 to electrically stimulate the at least one pelvic nerve 12 through the vaginal wall 14.

In some embodiments, the controller 40 may be wirelessly connected to a mobile communication device 42, such as a tablet, a mobile phone, a smart watch, or a computer, as shown in FIG. 2. The mobile communication device 42 may include a user interface that allows the user to control operation of the intravaginal electrical stimulation device 10. For example, the user may determine when the at least one electrically conductive electrode 20 is operating (i.e., when the at least one electrically conductive electrode 20 is electrically stimulating the at least one pelvic nerve 12). In this way, the user may choose to start the electrical stimulation based on when the user is feeling pain or discomfort.

The user may also determine a voltage, a current, an intensity, a duration, an amplitude, a frequency, and/or a duty cycle of the electrical stimulation. The user may also select a pain or discomfort level, and the pain or discomfort level may be associated with a particular stimulation pattern having a particular voltage, current, intensity, duration, amplitude, frequency, and/or duty cycle. The mobile communication device 42 may have a mobile application that allows the user to control the intravaginal electrical stimulation device 10. In some embodiments, the controller 40 and the mobile communication device 42 may be wirelessly connected via Bluetooth® or any other conventional wireless communication protocol. In some embodiments, artificial intelligence (AI) may be used to automatically adjust the electrical stimulation based on pain and/or patient preference.

In exemplary embodiments, a shape of the pulse delivered by the at least one electrically conductive electrode 20 is biphasic rectangular. In exemplary embodiments, a width of the pulse delivered by the at least one electrically conductive electrode 20 is about 50 to about 400 μs. A longer width may cause deeper activation for muscles. In exemplary embodiments, an amplitude of the pulse delivered by the at least one electrically conductive electrode 20 is about 0 to about 100 mA. A higher amplitude may cause motor control. In exemplary embodiments, a frequency of the pulse delivered by the at least one electrically conductive electrode 20 is about 1 to about 120 Hz. A lower frequency may be used for chronic pain, and a higher frequency may be used for acute pain. In exemplary embodiments, a duration of the session is about 15 to about 30 minutes. If the session is for overall nerve pain, a longer duration may be used.

In some embodiments, the user can, via the mobile communication device 42, alter any of the above parameters. As an example, the user can adjust the amplitude or another parameter via the mobile communication device 42 to cause motor control (i.e., muscle control) when the user is experiencing heavy menstrual bleeding. As another example, the controller 40 may use feedback from a sensor (for example, the sensor 825, as described in more detail below) to determine when to cause motor control. Data from the sensor (for example, the sensor 825) may indicate that the fluid-collection reservoir 30 is filling relatively quickly and, in response to receiving this data, the controller 40 may automatically adjust any parameters, such as the amplitude, of the electrical stimulation to cause motor control. This motor control may then decrease heavy menstrual bleeding experienced by the user.

The vaginal wall 14 with mucosal lining has a low impedance. If the impedance is incorrect, the intravaginal electrical stimulation device 10 may automatically shut off as a safety feature to avoid injury to the user (i.e., burns).

The mobile communication device 42 illustratively includes a memory 44 having programs for performing stimulation stored therein and a microprocessor 46 that executes the programs, as shown in FIG. 2. In some embodiments, the memory 44 and the microprocessor 46 may form part of the controller 40.

In some embodiments, the intravaginal electrical stimulation device 10 may be provided to the user with a deployment device 48, as shown in FIG. 3. The deployment device 48 holds the intravaginal electrical stimulation device 10 between two prongs 50 that extend outwardly from a body 52 of the deployment device 48. The two prongs 50 are configured to move relative to the body 52 in response to the user moving a button 54 coupled to the two prongs 50. As the user moves the button 54, the two prongs 50, and thus, the intravaginal electrical stimulation device 10, move with the button 54. In this way, the user may position the intravaginal electrical stimulation device 10 adjacent the vaginal canal 16 or inside of the vaginal canal 16 and use the deployment device 48 to insert the intravaginal electrical stimulation device 10 into the vaginal canal 16. The deployment device 48 helps the user orient the intravaginal electrical stimulation device 10 properly within the vaginal canal 16 so that the at least one electrically conductive electrode 20 is properly positioned adjacent the at least one pelvic nerve 12.

In some embodiments, the user may insert the intravaginal electrical stimulation device 10 without the use of the deployment device 48. For example, the open end of the menstrual collector 18 (at the opening 32) may be folded in on itself and held in a folded position by a user. Once inserted into the vaginal canal 16, the user releases the menstrual collector 18, and the menstrual collector 18 unfolds to a deployed position and nestles within the user's vaginal canal 16.

In some embodiments, the intravaginal electrical stimulation device 10 is reusable. In such an embodiment, the intravaginal electrical stimulation device 10 may be sanitized between uses. For example, the intravaginal electrical stimulation device 10 may be placed in boiling water, soaked in a vinegar solution, soaked in a hydrogen peroxide solution, placed in a commercial sterilizer that uses the microwave, washed in dish soap, etc. In some embodiments, the intravaginal electrical stimulation device 10 is provided for a single use.

In some embodiments, the menstrual collector 18 is formed of any one of or any combination of silicone (such as medical-grade silicone), polymer, rubber, latex, elastomer, or any other suitable material. In some embodiments, the at least one electrically conductive electrode 20 is formed of any biocompatible conductive material, such as gold.

Another embodiment of an intravaginal electrical stimulation device 210 is shown in FIGS. 4-6. The intravaginal electrical stimulation device 210 is substantially similar to the intravaginal electrical stimulation device 10 shown in FIGS. 1-3 and described herein. Accordingly, similar reference numbers in the 200 series indicate features that are common between the intravaginal electrical stimulation device 210 and the intravaginal electrical stimulation device 10. The description of the intravaginal electrical stimulation device 10 is incorporated by reference to apply to the intravaginal electrical stimulation device 210, expect in instances when it conflicts with the specific description and the drawings of the intravaginal electrical stimulation device 210.

The intravaginal electrical stimulation device 210 includes a menstrual collector 218 and at least one electrically conductive electrode 220, as shown in FIG. 4. The menstrual collector 218 is configured to be inserted into the vaginal canal 16 of the user. The at least one electrically conductive electrode 220 is coupled to the menstrual collector 218.

In some embodiments, the menstrual collector 218 is a menstrual cup, as shown in FIG. 6, or a menstrual disc. The menstrual collector 218 includes a body 222 and a rim 224. The body 222 is shaped to define an interior region 228 that provides a fluid-collection reservoir 230 to hold fluid therein. The rim 224 is coupled to the body 222 and formed to include an opening 232 into the interior region 228. In some embodiments, the menstrual collector 218 further includes a stem 226 coupled to the body 222 opposite the rim 224 at a bottom end 222B of the body 222, as shown in FIG. 5.

The body 222 defines an anterior side 222A (e.g., a front side) and a posterior side 222P (e.g., a back side), as shown in FIGS. 6 and 4. The anterior side 222A faces, engages, or contacts the anterior wall 14A of the vaginal canal 16. The posterior side 222P faces, engages, or contacts the posterior wall 14P of the vaginal canal 16.

The rim 224 of the menstrual collector 218 defines an upper end of the body 222, as shown in FIG. 5. In some embodiments, the rim 224 is angled, as shown in FIGS. 5 and 6. In other words, the rim 224 has a variable height from the bottom end 222B of the body 222 of the menstrual collector 218. An anterior portion 224A of the rim 224 at the anterior side 222A of the body 222 is located a first height H1 from the bottom end 222B of the body 222. A posterior portion 224P of the rim 224 at the posterior side 222P of the body 222 is located a second height H2 from the bottom end 222B of the body 222. In illustrative embodiments, the second height H2 is greater than the first height H1. The variable height enables the menstrual collector 218 to mimic the shape of the vaginal canal 16. In this way, the posterior portion 224P of the rim 224, in some embodiments, contacts or engages a portion of the cervix 15 while the menstrual collector 218 is located in the vaginal canal 16. In this embodiment, the rim 224 is not angled and does not have a variable height from the bottom end 222B of the body 222.

The stem 226 extends away from the body 222, as shown in FIG. 6. In some embodiments, the stem 226 is formed to include one or more ridges 234. In some embodiments, the stem 226 may be omitted, for example, if the menstrual collector 218 is a menstrual disc.

The at least one electrically conductive electrode 220 is coupled to the menstrual collector 218 such that, with the menstrual collector 218 inserted into the vaginal canal 16, at least a portion of the at least one electrically conductive electrode 220 engages or contacts the vaginal wall 14 of the vaginal canal 16. To this end, the at least one electrically conductive electrode 220 is coupled to an exterior surface of the menstrual collector 218. The at least one electrically conductive electrode 220 is configured to electrically stimulate the at least one pelvic nerve 12 through the vaginal wall 14 so that discomfort or pain associated with the pelvic condition or the procedure is alleviated.

In some embodiments, the at least one electrically conductive electrode 220 is coupled to the posterior side 222P of the body 222 of the menstrual collector 218, as shown in FIG. 4. In such an embodiment, the at least one electrically conductive electrode 220 engages or contacts the posterior wall 14P of the vaginal canal 16.

In some embodiments, the at least one electrically conductive electrode 220 is coupled to the anterior side 222A of the body 222 of the menstrual collector 218. In such an embodiment, the at least one electrically conductive electrode 220 engages or contacts the anterior wall 14A of the vaginal canal 16.

In some embodiments, the at least one electrically conductive electrode 220 is coupled to the posterior portion 224P of the rim 224 of the menstrual collector 218, as shown in FIG. 4. In such an embodiment, the at least one electrically conductive electrode 220 engages or contacts the cervix 15. In some embodiments, the at least one electrically conductive electrode 220 is coupled to the anterior portion 224A of the rim 224 of the menstrual collector 218.

In some embodiments, the at least one electrically conductive electrode 220 is coupled to one or more of the anterior side 222A of the body 222, the posterior side 222P of the body 222, the posterior portion 224P of the rim 224, and the anterior portion 224A of the rim 224. In some embodiments, the at least one electrically conductive electrode 220 includes more than one electrode, as shown in FIG. 4. The at least one electrically conductive electrode 220 may include any number of electrodes, such as two (2) electrodes to thirty (30) electrodes, including any specific number comprised therein.

In some embodiments, the at least one electrically conductive electrode 220 includes a plurality of rim electrodes 220R coupled to the rim 224 and spaced apart from one another about the rim 224, as shown in FIGS. 4 and 5. In some embodiments, the at least one electrically conductive electrode 220 includes a plurality of body electrodes 220B coupled to the body 222 of the menstrual collector 218, as shown in FIGS. 4 and 5.

The at least one electrically conductive electrode 220 electrically stimulates the at least one pelvic nerve 12 to modulate nerve activity and muscle response. Due to the shape of the menstrual collector 218 and the positioning of the at least one electrically conductive electrode 220 thereon, the electrical stimulation is directed to the pudendal nerve, for example. Placement of the plurality of rim electrodes 220R along the rim 224 and the plurality of body electrodes 220B on the posterior side 222P of the body 222 allow current to flow through tissues of the vaginal wall 14 where the pudendal nerve is located. The size, shape, and placement of the at least one electrically conductive electrode 220 may vary based on the pelvic nerve 12 being targeted, the pelvic condition, the procedure, and/or the user.

In some embodiments, the intravaginal electrical stimulation device 210 further includes a cord 236 and/or a battery 238, as shown in FIGS. 4 and 6. The cord 236 is coupled to the stem 226 or the bottom end 222B of the body 222, the battery 238, and the at least one electrically conductive electrode 220.

The cord 236 is coupled to the menstrual collector 218 such that, with the menstrual collector 218 inserted into the vaginal canal 16, at least a portion of the cord 236 is located exterior to the vaginal canal 16. The cord 236 extends between and interconnects the at least one electrically conductive electrode 220 and the battery 238, as shown in FIGS. 4 and 5.

The battery 238 is connected to the cord 236 such that, with the menstrual collector 218 inserted into the vaginal canal 16, the battery 238 is located exterior to the vaginal canal 16, as suggested in FIG. 4. In some embodiments, the cord 236 and the battery 238 may be located entirely within the vaginal canal 16. In some embodiments, the at least one electrically conductive electrode 220 may be wirelessly powered such that the cord 236 is omitted and the battery 238 is located exterior to the vaginal canal 16 or located within the vaginal canal 16.

In some embodiments, the intravaginal electrical stimulation device 210 further includes a controller 240, as shown in FIG. 6. The controller 240 selectively controls the at least one electrically conductive electrode 220 to electrically stimulate the at least one pelvic nerve 12 through the vaginal wall 14.

Another embodiment of an intravaginal electrical stimulation device 310 is shown in FIGS. 7-9. The intravaginal electrical stimulation device 310 is substantially similar to the intravaginal electrical stimulation device 10, 210 shown and described herein. Accordingly, similar reference numbers in the 300 series indicate features that are common between the intravaginal electrical stimulation device 310 and the intravaginal electrical stimulation device 10, 210. The description of the intravaginal electrical stimulation device 10, 210 is incorporated by reference to apply to the intravaginal electrical stimulation device 310, expect in instances when it conflicts with the specific description and the drawings of the intravaginal electrical stimulation device 310.

The intravaginal electrical stimulation device 310 includes a menstrual collector 318 and at least one electrically conductive electrode 320, as shown in FIG. 7. The menstrual collector 318 is configured to be inserted into the vaginal canal 16 of the user. The at least one electrically conductive electrode 320 is coupled to the menstrual collector 318.

In some embodiments, the menstrual collector 318 is a menstrual cup, as shown in FIG. 7, or a menstrual disc. The menstrual collector 318 includes a body 322 and a rim 324. The body 322 is shaped to define an interior region 328 that provides a fluid-collection reservoir 330 to hold fluid therein. The rim 324 is coupled to the body 322 and formed to include an opening 332 into the interior region 328. In some embodiments, the menstrual collector 318 includes a stem 326 coupled to the body 322 opposite the rim 324 at a bottom end 322B of the body 322, as shown in FIG. 8.

The body 322 defines an anterior side 322A (e.g., a front side) and a posterior side 322P (e.g., a back side), as shown in FIGS. 9 and 7. The anterior side 322A faces, engages, or contacts the anterior wall 14A of the vaginal canal 16. The posterior side 322P faces, engages, or contacts the posterior wall 14P of the vaginal canal 16.

As shown in FIGS. 8 and 9, the rim 324 is angled so as to have a variable height from the bottom end 322B of the body 322 of the menstrual collector 318. An anterior portion 324A of the rim 324 at the anterior side 322A of the body 322 is located a first height H1 from the bottom end 322B of the body 322. A posterior portion 324P of the rim 324 at the posterior side 322P of the body 322 is located a second height H2 from the bottom end 322B of the body 322. In illustrative embodiments, the second height H2 is greater than the first height H1. The variable height enables the menstrual collector 318 to mimic the shape of the vaginal canal 16. In this way, the posterior portion 324P of the rim 324, in some embodiments, contacts or engages a portion of the cervix 15 while the menstrual collector 318 is located in the vaginal canal 16.

The at least one electrically conductive electrode 320 is coupled to the menstrual collector 318 such that, with the menstrual collector 318 inserted into the vaginal canal 16, at least a portion of the at least one electrically conductive electrode 320 engages or contacts the vaginal wall 14 of the vaginal canal 16. In some embodiments, the at least one electrically conductive electrode 320 is coupled to the posterior side 322P of the body 322 of the menstrual collector 318, as shown in FIG. 7. In such an embodiment, the at least one electrically conductive electrode 320 engages or contacts the posterior wall 14P of the vaginal canal 16.

In some embodiments, the at least one electrically conductive electrode 320 is coupled to the anterior side 322A of the body 322 of the menstrual collector 318. In such an embodiment, the at least one electrically conductive electrode 320 engages or contacts the anterior wall 14A of the vaginal canal 16.

In some embodiments, the at least one electrically conductive electrode 320 is coupled to the posterior portion 324P of the rim 324 of the menstrual collector 318. In such an embodiment, the at least one electrically conductive electrode 320 engages or contacts the cervix 15. In some embodiments, the at least one electrically conductive electrode 320 is coupled to the anterior portion 324A of the rim 324 of the menstrual collector 318.

In some embodiments, the at least one electrically conductive electrode 320 is coupled to one or more of the anterior side 322A of the body 322, the posterior side 322P of the body 322, the posterior portion 324P of the rim 324, and the anterior portion 324A of the rim 324. In some embodiments, the at least one electrically conductive electrode 320 includes more than one electrode, as shown in FIG. 7. In such an embodiment, the at least one electrically conductive electrode 320 includes any number of electrodes, such as two (2) electrodes to thirty (30) electrodes, including any specific number comprised therein.

In some embodiments, the electrodes 320 cooperate to form a spoon-like shape adjacent the at least one pelvic nerve 12 and the cervicovaginal junction. The size, shape, and placement of the at least one electrically conductive electrode 320 may vary based on the pelvic nerve 12 being targeted, the pelvic condition, the procedure, and/or the user.

In some embodiments, the intravaginal electrical stimulation device 310 further includes a cord 336 and/or a battery 338, as shown in FIGS. 7 and 9. The cord 336 is coupled to the stem 326 or the bottom end 322B of the body 322, the battery 338, and the at least one electrically conductive electrode 320.

The cord 336 is coupled to the menstrual collector 318 such that, with the menstrual collector 318 inserted into the vaginal canal 16, at least a portion of the cord 336 is located exterior to the vaginal canal 16. The cord 336 extends between and interconnects the at least one electrically conductive electrode 320 and the battery 338, as suggested in FIG. 7.

The battery 338 is connected to the cord 336 such that, with the menstrual collector 318 inserted into the vaginal canal 16, the battery 338 is located exterior to the vaginal canal 16. In some embodiments, the cord 336 and the battery 338 may be located entirely within the vaginal canal 16. In some embodiments, the at least one electrically conductive electrode 320 may be wirelessly powered such that the cord 336 is omitted and the battery 338 is located exterior to the vaginal canal 16 or within the vaginal canal 16.

In some embodiments, the intravaginal electrical stimulation device 310 further includes a controller 340, as shown in FIG. 9. The controller 340 selectively controls the at least one electrically conductive electrode 320 to electrically stimulate the at least one pelvic nerve 12 through the vaginal wall 14.

Another embodiment of an intravaginal electrical stimulation device 410 is shown in FIGS. 10-12. The intravaginal electrical stimulation device 410 is substantially similar to the intravaginal electrical stimulation device 10, 210, 310 shown and described herein. Accordingly, similar reference numbers in the 400 series indicate features that are common between the intravaginal electrical stimulation device 410 and the intravaginal electrical stimulation device 10, 210, 310. The description of the intravaginal electrical stimulation device 10, 210, 310 is incorporated by reference to apply to the intravaginal electrical stimulation device 410, expect in instances when it conflicts with the specific description and the drawings of the intravaginal electrical stimulation device 410.

The intravaginal electrical stimulation device 410 includes a menstrual collector 418 and at least one electrically conductive electrode 420, as shown in FIG. 10. The menstrual collector 418 is configured to be inserted into the vaginal canal 16 of the user. The at least one electrically conductive electrode 420 is coupled to the menstrual collector 418.

In some embodiments, the menstrual collector 418 is a menstrual cup, as shown in FIG. 12, or a menstrual disc. The menstrual collector 418 includes a body 422 and a rim 424. The body 422 is shaped to define an interior region 428 that provides a fluid-collection reservoir 430 to hold fluid therein. The rim 424 is coupled to the body 422 and formed to include an opening 432 into the interior region 428. In some embodiments, the menstrual collector 418 further includes a stem 426 coupled to the body 422 opposite the rim 424 at a bottom end 422B of the body 422, as shown in FIG. 11. In some embodiments, the stem 426 may be omitted, for example, if the menstrual collector 418 is a menstrual disc.

The body 422 defines an anterior side 422A (e.g., a front side) and a posterior side 422P (e.g., a back side), as shown in FIGS. 12 and 10. The anterior side 422A faces, engages, or contacts the anterior wall 14A of the vaginal canal 16. The posterior side 422P faces, engages, or contacts the posterior wall 14P of the vaginal canal 16.

As shown in FIGS. 11 and 12, the rim 424 is angled so as to have a variable height from the bottom end 422B of the body 422 of the menstrual collector 418. An anterior portion 424A of the rim 424 at the anterior side 422A of the body 422 is located a first height H1 from the bottom end 422B of the body 422. A posterior portion 424P of the rim 424 at the posterior side 422P of the body 422 is located a second height H2 from the bottom end 422B of the body 422. In illustrative embodiments, the second height H2 is greater than the first height H1. The variable height enables the menstrual collector 418 to mimic the shape of the vaginal canal 16. In this way, the posterior portion 424P of the rim 424, in some embodiments, contacts or engages a portion of the cervix 15 while the menstrual collector 418 is located in the vaginal canal 16.

The at least one electrically conductive electrode 420 is coupled to the menstrual collector 418 such that, with the menstrual collector 418 inserted into the vaginal canal 16, at least a portion of the at least one electrically conductive electrode 420 engages or contacts the vaginal wall 14 of the vaginal canal 16. In some embodiments, the at least one electrically conductive electrode 420 is coupled to the posterior side 422P of the body 422 of the menstrual collector 418, as shown in FIG. 10. In such an embodiment, the at least one electrically conductive electrode 420 engages or contacts the posterior wall 14P of the vaginal canal 16.

In some embodiments, the at least one electrically conductive electrode 420 is coupled to the anterior side 422A of the body 422 of the menstrual collector 418. In such an embodiment, the at least one electrically conductive electrode 420 engages or contacts the anterior wall 14A of the vaginal canal 16.

In some embodiments, the at least one electrically conductive electrode 420 is coupled to the posterior portion 424P of the rim 424 of the menstrual collector 418. In such an embodiment, the at least one electrically conductive electrode 420 engages or contacts the cervix 15. In some embodiments, the at least one electrically conductive electrode 420 is coupled to the anterior portion 424A of the rim 424 of the menstrual collector 418.

In some embodiments, the at least one electrically conductive electrode 420 is coupled to one or more of the anterior side 422A of the body 422, the posterior side 422P of the body 422, the posterior portion 424P of the rim 424, and the anterior portion 424A of the rim 424. In some embodiments, the at least one electrically conductive electrode 420 includes more than one electrode, as shown in FIG. 10. In such an embodiment, the at least one electrically conductive electrode 420 may include any number of electrodes, such as two (2) electrodes to thirty (30) electrodes, including any specific number comprised therein.

In some embodiments, the at least one electrically conductive electrode 420 includes a plurality of exterior body electrodes 420E coupled to an exterior of the body 422 of the menstrual collector 418, as shown in FIGS. 10 and 11. In such an embodiment, the plurality of exterior body electrodes 420E is illustratively coupled to the posterior side 422P of the body 422. In some embodiments, the plurality of exterior body electrodes 420E may be coupled to the body 422 to form a V-shape.

In some embodiments, the at least one electrically conductive electrode 420 further includes a plurality of interior body electrodes 4201 coupled to an interior of the body 422 of the menstrual collector 418, as shown in FIG. 12. In such an embodiment, the plurality of interior body electrodes 4201 is configured to engage or contact a portion of the cervix 15. In some embodiments, the plurality of interior body electrodes 4201 may be coupled to the body 422 to form a V-shape.

In some embodiments, the intravaginal electrical stimulation device 410 further includes a cord 436 and/or a battery 438, as shown in FIGS. 10 and 12. The cord 436 is coupled to the stem 426 or the bottom end 422B of the body 422, the battery 438, and the at least one electrically conductive electrode 420.

In some embodiments, the intravaginal electrical stimulation device 410 further includes a controller 440, as shown in FIG. 12. The controller 440 selectively controls the at least one electrically conductive electrode 420 to electrically stimulate the at least one pelvic nerve 12 through the vaginal wall 14.

Another embodiment of an intravaginal electrical stimulation device 510 is shown in FIGS. 13-15. The intravaginal electrical stimulation device 510 is substantially similar to the intravaginal electrical stimulation device 10, 210, 310, 410 shown and described herein. Accordingly, similar reference numbers in the 500 series indicate features that are common between the intravaginal electrical stimulation device 510 and the intravaginal electrical stimulation device 10, 210, 310, 410. The description of the intravaginal electrical stimulation device 10, 210, 310, 410 is incorporated by reference to apply to the intravaginal electrical stimulation device 510, expect in instances when it conflicts with the specific description and the drawings of the intravaginal electrical stimulation device 510.

The intravaginal electrical stimulation device 510 includes a menstrual collector 518 and at least one electrically conductive electrode 520, as shown in FIG. 13. The menstrual collector 518 is configured to be inserted into the vaginal canal 16 of the user. The at least one electrically conductive electrode 520 is coupled to the menstrual collector 518.

In some embodiments, the menstrual collector 518 is a menstrual cup, as shown in FIG. 15, or a menstrual disc. The menstrual collector 518 includes a body 522 and a rim 524. The body 522 is shaped to define an interior region 528 that provides a fluid-collection reservoir 530 to hold fluid therein. The rim 524 is coupled to the body 522 and formed to include an opening 532 into the interior region 528. In some embodiments, the menstrual collector 518 further includes a stem 526 coupled to the body 522 opposite the rim 524 at a bottom end 522B of the body 522, as shown in FIG. 14.

The body 522 defines an anterior side 522A (e.g., a front side) and a posterior side 522P (e.g., a back side), as shown in FIGS. 15 and 13. The anterior side 522A faces, engages, or contacts the anterior wall 14A of the vaginal canal 16. The posterior side 522P faces, engages, or contacts the posterior wall 14P of the vaginal canal 16.

As shown in FIGS. 14 and 15, the rim 524 is angled so as to have a variable height from the bottom end 522B of the body 522 of the menstrual collector 518. An anterior portion 524A of the rim 524 at the anterior side 522A of the body 522 is located a first height H1 from the bottom end 522B of the body 522. A posterior portion 524P of the rim 524 at the posterior side 522P of the body 522 is located a second height H2 from the bottom end 522B of the body 522. In illustrative embodiments, the second height H2 is greater than the first height H1. The variable height enables the menstrual collector 518 to mimic the shape of the vaginal canal 16. In this way, the posterior portion 524P of the rim 524, in some embodiments, contacts or engages a portion of the cervix 15 while the menstrual collector 518 is located in the vaginal canal 16.

The at least one electrically conductive electrode 520 is coupled to the menstrual collector 518 such that, with the menstrual collector 518 inserted into the vaginal canal 16, at least a portion of the at least one electrically conductive electrode 520 engages or contacts the vaginal wall 14 of the vaginal canal 16. In some embodiments, the at least one electrically conductive electrode 520 is coupled to the posterior side 522P of the body 522 of the menstrual collector 518, as shown in FIG. 13. In such an embodiment, the at least one electrically conductive electrode 520 engages or contacts the posterior wall 14P of the vaginal canal 16.

In some embodiments, the at least one electrically conductive electrode 520 is coupled to the anterior side 522A of the body 522 of the menstrual collector 518. In such an embodiment, the at least one electrically conductive electrode 520 engages or contacts the anterior wall 14A of the vaginal canal 16.

In some embodiments, the at least one electrically conductive electrode 520 is coupled to the posterior portion 524P of the rim 524 of the menstrual collector 518, as shown in FIG. 13. In such an embodiment, the at least one electrically conductive electrode 520 engages or contacts the cervix 15. In some embodiments, the at least one electrically conductive electrode 520 is coupled to the anterior portion 524A of the rim 524 of the menstrual collector 518, as shown in FIG. 15.

In some embodiments, the at least one electrically conductive electrode 520 is coupled to one or more of the anterior side 522A of the body 522, the posterior side 522P of the body 522, the posterior portion 524P of the rim 524, and the anterior portion 524A of the rim 524. In some embodiments, the at least one electrically conductive electrode 520 includes more than one electrode, as shown in FIG. 14. In such an embodiment, the at least one electrically conductive electrode 520 may include any number of electrodes, such as two (2) electrodes to thirty (30) electrodes, including any specific number comprised therein.

In some embodiments, the at least one electrically conductive electrode 520 includes a plurality of rim electrodes 520R coupled to the rim 524 and spaced apart from one another about the rim 524, as shown in FIGS. 13 and 14. In this way, the plurality of rim electrodes 520R is located adjacent the cervicovaginal junction. In some embodiments, the at least one electrically conductive electrode 520 includes at least one body electrode 520B coupled to the body 522 of the menstrual collector 518, as shown in FIGS. 13 and 14.

In some embodiments, the intravaginal electrical stimulation device 510 includes a cord 536 and/or a battery 538, as shown in FIG. 15. The cord 536 extends between and interconnects the at least one electrically conductive electrode 520 and the battery 538. The cord 536 and the battery 538 may be located entirely within the vaginal canal 16, as suggested in FIG. 15. In some embodiments, the at least one electrically conductive electrode 520 may be wirelessly powered such that the cord 536 is omitted. In some embodiments, the cord 536 and the battery 538 may be located exterior to the vaginal canal 16, as previously described.

In some embodiments, the intravaginal electrical stimulation device 510 further includes a controller 540, as shown in FIG. 15. The controller 540 selectively controls the at least one electrically conductive electrode 520 to electrically stimulate the at least one pelvic nerve 12 through the vaginal wall 14.

Another embodiment of an intravaginal electrical stimulation device 610 is shown in FIGS. 16-18. The intravaginal electrical stimulation device 610 is substantially similar to the intravaginal electrical stimulation device 10, 210, 310, 410, 510 shown and described herein. Accordingly, similar reference numbers in the 600 series indicate features that are common between the intravaginal electrical stimulation device 610 and the intravaginal electrical stimulation device 10, 210, 310, 410, 510. The description of the intravaginal electrical stimulation device 10, 210, 310, 410, 510 is incorporated by reference to apply to the intravaginal electrical stimulation device 610, expect in instances when it conflicts with the specific description and the drawings of the intravaginal electrical stimulation device 610.

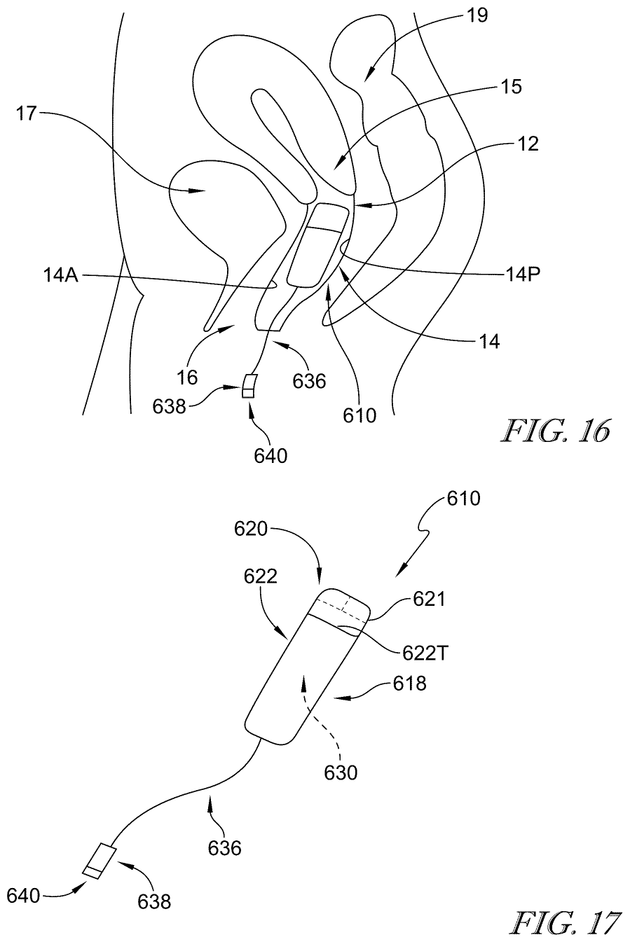

The intravaginal electrical stimulation device 610 includes a menstrual collector 618 and at least one electrically conductive electrode 620, as shown in FIG. 17. The menstrual collector 618 is configured to be inserted into the vaginal canal 16 of the user. The at least one electrically conductive electrode 620 is coupled to the menstrual collector 618.

In some embodiments, the menstrual collector 18 is a menstrual plug, as shown in FIG. 17. The menstrual plug may also be referred to as a tampon. The menstrual collector 618 includes a body 622 defining a fluid-collection reservoir 630 to absorb fluid therein.

The fluid-collection reservoir 630 of the body 622 may absorb fluid, such as menstrual fluid. In this way, the intravaginal electrical stimulation device 610 may be used by the user during menstruation. The fluid-collection reservoir 630 of the body 622 may absorb about 6 grams of fluid to about 18 grams of fluid without leaking of the fluid out of the body 622. The fluid-collection reservoir 630 of the body 622 may provide absorbency of a light-absorbency tampon (6 grams of fluid or less), a regular-absorbency tampon (6 to 9 grams of fluid), a super-absorbency tampon (9 to 12 grams of fluid), a super plus-absorbency tampon (12 to 15 grams of fluid), or an ultra-absorbency tampon (15 to 18 grams of fluid). The intravaginal electrical stimulation device 610 may also be used outside of menstruation.

The at least one electrically conductive electrode 620 is coupled to the menstrual collector 618 such that, with the menstrual collector 618 inserted into the vaginal canal 16, at least a portion of the at least one electrically conductive electrode 620 engages or contacts the vaginal wall 14 of the vaginal canal 16, as suggested in FIG. 16. In some embodiments, the at least one electrically conductive electrode 620 is housed in a silicone scaffold 621, as shown in FIG. 17. The silicone scaffold 621 has natural elastic tendencies and, thus, will spring open behind the cervix 15, thereby cradling the uterosacral ligaments.

In some embodiments, the silicone scaffold 621 is coupled to a top surface 622T of the body 622 of the menstrual collector 618, as shown in FIG. 17. In such an embodiment, the at least one electrically conductive electrode 620 engages or contacts the anterior wall 14A of the vaginal canal 16, the posterior wall 14P of the vaginal canal 16, and/or the cervix 15.

In some embodiments, the at least one electrically conductive electrode 620 includes more than one electrode. In such an embodiment, the at least one electrically conductive electrode 620 may include any number of electrodes, such as two (2) electrodes to thirty (30) electrodes, including any specific number comprised therein.

In some embodiments, the intravaginal electrical stimulation device 610 further includes a cord 636 and/or a battery 638, as shown in FIGS. 16 and 17. The cord 636 is coupled to the body 622, the battery 638, and the at least one electrically conductive electrode 620. The cord 636 may also be used to remove the menstrual collector 618 from the vaginal canal 16.

The cord 636 is coupled to the menstrual collector 618 such that, with the menstrual collector 618 inserted into the vaginal canal 16, at least a portion of the cord 636 is located exterior to the vaginal canal 16, as shown in FIG. 16. The cord 636 extends between and interconnects the at least one electrically conductive electrode 620 and the battery 638. The battery 638 is connected to the cord 636 such that, with the menstrual collector 618 inserted into the vaginal canal 16, the battery 638 is located exterior to the vaginal canal 16, as shown in FIG. 16.

In some embodiments, the intravaginal electrical stimulation device 610 further includes a controller 640, as shown in FIG. 17. The controller 640 selectively controls the at least one electrically conductive electrode 620 to electrically stimulate the at least one pelvic nerve 12 through the vaginal wall 14. The at least one pelvic nerve 12 may be a hypogastric nerve, a pudendal nerve, or any other nerve.

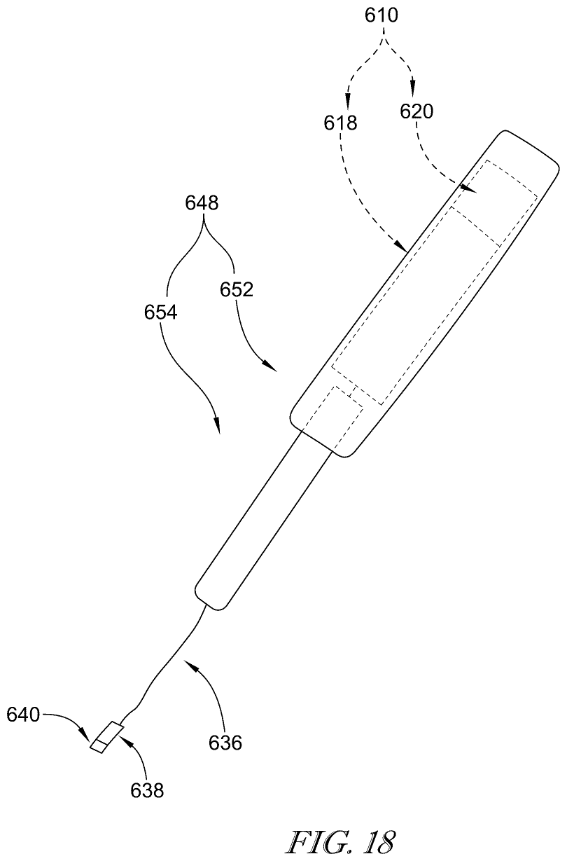

In some embodiments, the intravaginal electrical stimulation device 610 may be provided to the user with a deployment device 648, as shown in FIG. 18. The deployment device 648 includes an outer case 652 and an applicator 654. The intravaginal electrical stimulation device 610 is located inside of the outer case 652, and the applicator 654 abuts a bottom end of the intravaginal electrical stimulation device 610. The applicator 654 is movable relative to the outer case 652 to push the intravaginal electrical stimulation device 610 upwardly out of the outer case 652 and into position in the vaginal canal 16. The applicator 654 illustratively has a hole extending therethrough so that the cord 636 and the battery 638 may pass through the applicator 654.

The user may position the outer case 652 adjacent the vaginal canal 16 or within the vaginal canal 16. The outer case 652 may be formed to include a notch that indicates directionality for the initial insertion to ensure the intravaginal electrical stimulation device 610 is placed correctly. Then, the user can apply force to a bottom end of the applicator 654 thereby moving the applicator 654 upwardly into the outer case 652. The upward movement of the applicator 654 causes movement of the intravaginal electrical stimulation device 610 out of the outer case 652 and into the vaginal canal 16. The user may then move the deployment device 648 away from the vaginal canal 16. Once in the vaginal canal 16, the intravaginal electrical stimulation device 610 is free to expand.

In some embodiments, the user may insert the intravaginal electrical stimulation device 610 without the use of the deployment device 648. In some embodiments, the intravaginal electrical stimulation device 610 or portions thereof (such as the at least one electrically conductive electrode 620, the silicone scaffold 621, the cord 636, the battery 638, and/or the controller 640) are reusable. In some embodiments, the intravaginal electrical stimulation device 610 is provided for a single use.

In some embodiments, the menstrual collector 618 is formed of any one of or any combination of cotton, rayon, polyester, polyethylene, polypropylene, fiber finishes, or any other suitable material. In some embodiments, the at least one electrically conductive electrode 620 is formed of any biocompatible conductive material, such as gold.

Another embodiment of an intravaginal electrical stimulation device 710 is shown in FIG. 19. The intravaginal electrical stimulation device 710 is substantially similar to the intravaginal electrical stimulation device 10, 210, 310, 410, 510, 610 shown and described herein. Accordingly, similar reference numbers in the 700 series indicate features that are common between the intravaginal electrical stimulation device 710 and the intravaginal electrical stimulation device 10, 210, 310, 410, 510, 610. The description of the intravaginal electrical stimulation device 10, 210, 310, 410, 510, 610 is incorporated by reference to apply to the intravaginal electrical stimulation device 710, expect in instances when it conflicts with the specific description and the drawings of the intravaginal electrical stimulation device 710.

The intravaginal electrical stimulation device 710 includes a vaginal insert 718 and at least one electrically conductive electrode 720, as shown in FIG. 19. The vaginal insert 718 is configured to be inserted into the vaginal canal 16 of the user. The at least one electrically conductive electrode 720 is coupled to the vaginal insert 718.

In illustrative embodiments, the vaginal insert 718 is not configured to hold, collect, or absorb fluid therein. The vaginal insert 718 includes a first arm 722, a second arm 724, and a stem 726. The first arm 722, the second arm 724, and the stem 726 cooperate to form a Y-shape. However, any other shape is contemplated.

An exterior surface of the first arm 722 faces, engages, or contacts the anterior wall 14A of the vaginal canal 16. An exterior surface of the second arm 724 faces, engages, or contacts the posterior wall 14P of the vaginal canal 16. In some embodiments, an interior surface of the first arm 722 (that faces toward the second arm 724) and/or an interior surface of the second arm 724 (that faces toward the first arm 722) contacts or engages a portion of the cervix 15 while the vaginal insert 718 is located in the vaginal canal 16.

The at least one electrically conductive electrode 720 is coupled to the vaginal insert 718 such that, with the vaginal insert 718 inserted into the vaginal canal 16, at least a portion of the at least one electrically conductive electrode 720 engages or contacts the vaginal wall 14 of the vaginal canal 16 and/or the cervix 15. In some embodiments, the at least one electrically conductive electrode 720 includes a plurality of electrodes 720 coupled to the exterior surface of the first arm 722, the exterior surface of the second arm 724, the interior surface of the first arm 722, and/or the interior surface of the second arm 724, as shown in FIG. 19. In such an embodiment, the plurality of electrodes 720 engage or contact the anterior wall 14A of the vaginal canal 16, the posterior wall 14P of the vaginal canal 16, and/or the cervix 15. The plurality of electrodes 720 may include any number of electrodes, such as two (2) electrodes to thirty (30) electrodes, including any specific number comprised therein.

In some embodiments, the intravaginal electrical stimulation device 710 further includes a cord 736 and/or a battery 738, as shown in FIG. 19. The cord 736 is coupled to the stem 726, the battery 738, and the at least one electrically conductive electrode 720.

In some embodiments, the intravaginal electrical stimulation device 710 further includes a controller 740, as shown in FIG. 19. The controller 740 selectively controls the at least one electrically conductive electrode 720 to electrically stimulate the at least one pelvic nerve 12.

Another embodiment of an intravaginal electrical stimulation device 710′ is shown in FIG. 19A. The intravaginal electrical stimulation device 710′ is substantially similar to the intravaginal electrical stimulation device 10, 210, 310, 410, 510, 610, 710 shown and described herein. Accordingly, similar reference numbers in the 700 series with a prime symbol indicate features that are common between the intravaginal electrical stimulation device 710′ and the intravaginal electrical stimulation device 10, 210, 310, 410, 510, 610, 710. The description of the intravaginal electrical stimulation device 10, 210, 310, 410, 510, 610, 710 is incorporated by reference to apply to the intravaginal electrical stimulation device 710′, expect in instances when it conflicts with the specific description and the drawings of the intravaginal electrical stimulation device 710′.

The intravaginal electrical stimulation device 710′ includes a vaginal insert 718′ and at least one electrically conductive electrode 720′, as shown in FIG. 19A. The vaginal insert 718′ is configured to be inserted into the vaginal canal 16 of the user. The at least one electrically conductive electrode 720′ is coupled to the vaginal insert 718′.

In illustrative embodiments, the vaginal insert 718′ is not configured to hold, collect, or absorb fluid therein. The vaginal insert 718′ may be a balloon or a condom. For example, the vaginal insert 718′ may be inserted into the vaginal canal 16 and then expanded. An another example, the vaginal insert 718′ may be inserted onto an ultrasound probe or a speculum and then inserted into the vaginal canal 16.

The at least one electrically conductive electrode 720′ is coupled to the vaginal insert 718′ such that, with the vaginal insert 718′ inserted into the vaginal canal 16, at least a portion of the at least one electrically conductive electrode 720′ engages or contacts the vaginal wall 14 of the vaginal canal 16 and/or the cervix 15. In some embodiments, the at least one electrically conductive electrode 720′ includes a plurality of electrodes 720′ coupled to the exterior surface of the vaginal insert 718′, as shown in FIG. 19A. The plurality of electrodes 720′ may include any number of electrodes, such as two (2) electrodes to thirty (30) electrodes, including any specific number comprised therein.

Another embodiment of an intravaginal electrical stimulation device 710″ is shown in FIG. 19B. The intravaginal electrical stimulation device 710″ is substantially similar to the intravaginal electrical stimulation device 10, 210, 310, 410, 510, 610, 710, 710′ shown and described herein. Accordingly, similar reference numbers in the 700 series with a double prime symbol indicate features that are common between the intravaginal electrical stimulation device 710″ and the intravaginal electrical stimulation device 10, 210, 310, 410, 510, 610, 710, 710′. The description of the intravaginal electrical stimulation device 10, 210, 310, 410, 510, 610, 710, 710′ is incorporated by reference to apply to the intravaginal electrical stimulation device 710″, expect in instances when it conflicts with the specific description and the drawings of the intravaginal electrical stimulation device 710″.

The intravaginal electrical stimulation device 710″ includes a vaginal insert 718″ and at least one electrically conductive electrode 720″, as shown in FIG. 19B. The vaginal insert 718″ is configured to be inserted into the vaginal canal 16 of the user. The at least one electrically conductive electrode 720″ is coupled to the vaginal insert 718″.

In illustrative embodiments, the vaginal insert 718″ is not configured to hold, collect, or absorb fluid therein. The vaginal insert 718″ may be a ring. In this way, other devices may pass through the vaginal insert 718″ while the vaginal insert 718″ is inserted into the vaginal canal 16. For example, the vaginal insert 718″ may be inserted into the vaginal canal 16 and then an ultrasound probe, a speculum, or other similar device may be inserted into the vaginal canal 16 through the vaginal insert 718″ for the procedure so that the intravaginal electrical stimulation device 710″ and the ultrasound probe, the speculum, or the other similar device may be used simultaneously during the procedure.

The at least one electrically conductive electrode 720″ is coupled to the vaginal insert 718″ such that, with the vaginal insert 718″ inserted into the vaginal canal 16, at least a portion of the at least one electrically conductive electrode 720″ engages or contacts the vaginal wall 14 of the vaginal canal 16 and/or the cervix 15. In some embodiments, the at least one electrically conductive electrode 720″ includes a plurality of electrodes 720″ coupled to the exterior surface of the vaginal insert 718″, as shown in FIG. 19B. The plurality of electrodes 720″ may include any number of electrodes, such as two (2) electrodes to thirty (30) electrodes, including any specific number comprised therein.

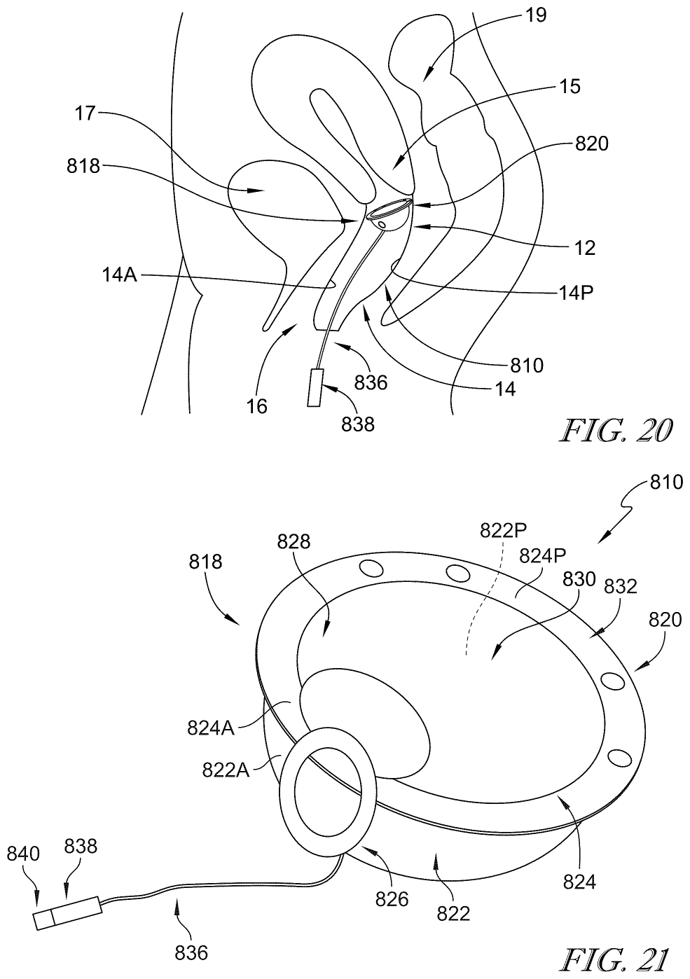

Another embodiment of an intravaginal electrical stimulation device 810 is shown in FIGS. 20-22. The intravaginal electrical stimulation device 810 is substantially similar to the intravaginal electrical stimulation device 10, 210, 310, 410, 510, 610, 710, 710′, 710″ shown and described herein. Accordingly, similar reference numbers in the 800 series indicate features that are common between the intravaginal electrical stimulation device 810 and the intravaginal electrical stimulation device 10, 210, 310, 410, 510, 610, 710, 710′, 710″. The description of the intravaginal electrical stimulation device 10, 210, 310, 410, 510, 610, 710, 710′, 710″ is incorporated by reference to apply to the intravaginal electrical stimulation device 810, expect in instances when it conflicts with the specific description and the drawings of the intravaginal electrical stimulation device 810.

The intravaginal electrical stimulation device 810 includes a menstrual collector 818 and at least one electrically conductive electrode 820, as shown in FIG. 21. The menstrual collector 818 is configured to be inserted into the vaginal canal 16 of the user. The at least one electrically conductive electrode 820 is coupled to the menstrual collector 818 such that the at least one electrically conductive electrode 820 is located within the vaginal canal 16 while the menstrual collector 818 is located within the vaginal canal 16.

In some embodiments, the menstrual collector 818 is a menstrual disc, as shown in FIG. 21, or a menstrual cup. The menstrual collector 818 includes a body 822 and a rim 824. The body 822 is shaped to define an interior region 828 that provides a fluid-collection reservoir 830 to hold fluid therein. The rim 824 is coupled to the body 822 and formed to include an opening 832 into the interior region 828.

In some embodiments, the menstrual collector 818 further includes a removal tab 826, as shown in FIG. 21. The removal tab 826 is coupled to the body 822. The removal tab 826 is illustratively formed as a full-circle loop or ring that is coupled to the body 822 at one end thereof and free from the body 822 at an opposite end thereof. In some embodiments, the removal tab 826 is omitted. In some embodiments, the removal tab 826 is omitted and/or replaced with a removal cord 827 that the user may pull to remove the menstrual collector 818 from the vaginal canal 16, as shown in FIG. 24. The removal cord 827 may extend around the rim 824, as shown in FIG. 24, and exterior to the vaginal canal 16. When pulled by the user, the removal cord 827 may tighten or cinch the rim 824 so that fluid within the menstrual collector 818 is substantially trapped in the menstrual collector 818 during removal. For example, when pulled by the user, the removal cord 827 may cause the rim 824 to close or be drawn together.

The body 822 defines an anterior side 822A (e.g., a front side) and a posterior side 822P (e.g., a back side), as shown in FIG. 21. The anterior side 822A faces, engages, or contacts an anterior wall 14A of the vaginal canal 16 located adjacent a bladder 17 of the user. The posterior side 822P faces, engages, or contacts a posterior wall 14P of the vaginal canal 16 located adjacent a rectum 19 of the user.

In some embodiments, as shown in FIG. 24, the body 822 includes measurement lines 823 on an exterior and/or an interior of the body 822. The measurement lines 823 inform the user regarding an amount of fluid in the fluid-collection reservoir 830. For example, each of the measurement lines 823 may be related to a level of menstrual bleeding. One of the measurement lines 823 may indicate a light flow, while another of the measurement lines 823 may indicate a heavy flow.

In some embodiments, as shown in FIG. 24, the intravaginal electrical stimulation device 810 further includes a sensor 825 configured to determine the amount of fluid in the fluid-collection reservoir 830. The sensor 825 may be in communication with the controller 840. In this way, the user may be informed regarding the amount of fluid in the fluid-collection reservoir 830 and/or a rate of fluid flow into the fluid-collection reservoir 830. A notification or an alert may be given to the user on the mobile communication device 42 based on data from the sensor 825. For example, if the fluid-collection reservoir 830 is substantially full, the user may receive a notification that the menstrual collector 818 needs to be emptied. As another example, if the fluid-collection reservoir 830 is filling relatively quickly, the user may receive an alert that medical assistance should be sought due to heavy menstrual bleeding.

The rim 824 of the menstrual collector 818 defines an upper end of the body 822, as shown in FIG. 21. The rim 824 defines an anterior portion 824A at the anterior side 822A of the body 822 and a posterior portion 824P at the posterior side 822P of the body 822.

The at least one electrically conductive electrode 820 is coupled to the menstrual collector 818 such that, with the menstrual collector 818 inserted into the vaginal canal 16, at least a portion of the at least one electrically conductive electrode 820 engages or contacts the vaginal wall 14 of the vaginal canal 16, as suggested in FIG. 20. In some embodiments, the at least one electrically conductive electrode 820 is coupled to the rim 824 of the menstrual collector 818. In such an embodiment, the at least one electrically conductive electrode 820 engages or contacts the vaginal wall 14 of the vaginal canal 16.

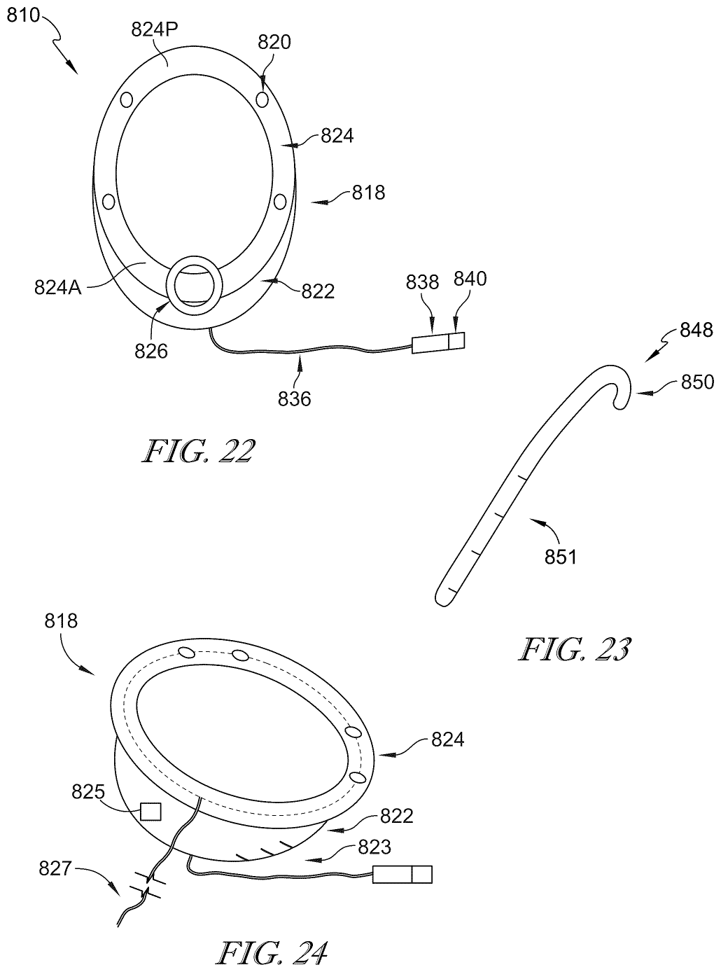

As an example, the at least one electrically conductive electrode 820 may include four electrodes 820 spaced apart from one another about the rim 824, as shown in FIG. 21. FIG. 21 shows the menstrual collector 818 while the menstrual collector 818 is not inserted into the vaginal canal 16 (i.e., while the menstrual collector 818 is not deformed to fit within the vaginal canal 16). FIG. 22 shows the menstrual collector 818 while the menstrual collector 818 is inserted into the vaginal canal 16 (i.e., while the menstrual collector 818 is deformed to fit within the vaginal canal 16). While inserted, the electrodes 820 may be located at a 2 o'clock position, a 4 o'clock position, an 8 o'clock position, and a 10 o'clock position around the rim 824 to contact the vaginal wall 14 and the at least one pelvic nerve 12, as shown in FIG. 22.

In some embodiments, the at least one electrically conductive electrode 820 is coupled to one or more of the anterior side 822A of the body 822, the posterior side 822P of the body 822, the posterior portion 824P of the rim 824, and the anterior portion 824A of the rim 824. In some embodiments, the at least one electrically conductive electrode 820 includes more than one electrode. In such an embodiment, the at least one electrically conductive electrode 820 includes any number of electrodes, such as two (2) electrodes to thirty (30) electrodes, including any specific number comprised therein. The size, shape, and placement of the at least one electrically conductive electrode 820 may vary based on the at least one pelvic nerve 12 being targeted, the pelvic condition, the procedure, and/or the user.

In some embodiments, the intravaginal electrical stimulation device 810 further includes a cord 836 and/or a battery 838, as shown in FIGS. 20 and 21. The cord 836 is coupled to the battery 838 and the at least one electrically conductive electrode 820.

The cord 836 is coupled to the menstrual collector 818 such that, with the menstrual collector 818 inserted into the vaginal canal 16, at least a portion of the cord 836 is located exterior to the vaginal canal 16, as shown in FIG. 20. The battery 838 is connected to the cord 836 such that, with the menstrual collector 818 inserted into the vaginal canal 16, the battery 838 is located exterior to the vaginal canal 16, as shown in FIG. 20.

In some embodiments, the intravaginal electrical stimulation device 810 further includes a controller 840, as shown in FIG. 21. The controller 840 is in communication with the at least one electrically conductive electrode 820 to send electrical signals thereto. The controller 840 selectively controls the at least one electrically conductive electrode 820 to electrically stimulate the at least one pelvic nerve 12 through the vaginal wall 14.

In some embodiments, the intravaginal electrical stimulation device 810 may be provided to the user with a deployment device 848, as shown in FIG. 23. The deployment device 848 helps the user insert and/or remove the intravaginal electrical stimulation device 810. The deployment device 848 helps the user orient the intravaginal electrical stimulation device 810 properly within the vaginal canal 16 so that the at least one electrically conductive electrode 820 is properly positioned adjacent the at least one pelvic nerve 12. The deployment device 848 illustratively includes a hook 850 that grabs onto the removal tab 826. The deployment device 848 further includes measurement guidelines 851 that help the user determine what size of the menstrual collector 818 to use. For example, the user may insert the deployment device 848 into the vaginal canal 16 to determine how high up the cervix 15 is located. One or more of the measurement guidelines 851 will be visible exterior to the vaginal canal 16, which will indicate to the user which size of the menstrual collector 818 to use (for example, small, medium, or large). In some embodiments, the user may insert and/or remove the intravaginal electrical stimulation device 810 without the use of the deployment device 848.

1. An intravaginal electrical stimulation device comprising
a menstrual collector configured to be inserted into a vaginal canal of a user, the menstrual collector defining a fluid-collection reservoir to hold or absorb fluid therein, and
at least one electrically conductive electrode coupled to the menstrual collector such that, with the menstrual collector inserted into the vaginal canal, at least a portion of the at least one electrically conductive electrode is arranged to contact a vaginal wall of the vaginal canal, the at least one electrically conductive electrode configured to electrically stimulate at least one pelvic nerve through the vaginal wall so that discomfort or pain associated with a pelvic condition or a procedure is alleviated.

2. The intravaginal electrical stimulation device of claim 1, wherein the menstrual collector includes a body shaped to define an interior region that provides the fluid-collection reservoir and a rim coupled to the body and formed to include an opening into the interior region.

3. The intravaginal electrical stimulation device of claim 2, wherein the rim of the menstrual collector includes an anterior portion and a posterior portion opposite the anterior portion, and wherein the anterior portion of the rim is a first height from a bottom end of the body of the menstrual collector and the posterior portion of the rim is a second height from the bottom end of the body, the second height being greater than the first height to conform to a shape of the vaginal canal.

4. The intravaginal electrical stimulation device of claim 3, wherein the posterior portion of the rim is configured to engage a cervix of the user while the menstrual collector is located in the vaginal canal.

5. The intravaginal electrical stimulation device of claim 3, wherein the at least one electrically conductive electrode includes a first electrode coupled to the rim of the menstrual collector and a second electrode coupled to a posterior side of the body of the menstrual collector that is arranged to contact the vaginal wall, and wherein the vaginal wall is a posterior wall of the vaginal canal.

6. The intravaginal electrical stimulation device of claim 2, wherein the at least one electrically conductive electrode includes a plurality of rim electrodes coupled to the rim and spaced apart from one another about the rim of the menstrual collector.

7. The intravaginal electrical stimulation device of claim 6, wherein the at least one electrically conductive electrode further includes a plurality of body electrodes coupled to the body of the menstrual collector.

8. The intravaginal electrical stimulation device of claim 2, wherein the at least one electrically conductive electrode is coupled to a posterior side of the body of the menstrual collector that is arranged to contact the vaginal wall, and wherein the vaginal wall is a posterior wall of the vaginal canal.

9. The intravaginal electrical stimulation device of claim 1, further comprising a controller in communication with the at least one electrically conductive electrode and configured to selectively control the at least one electrically conductive electrode to electrically stimulate the at least one pelvic nerve through the vaginal wall.

10. The intravaginal electrical stimulation device of claim 1, further comprising a cord connected to the at least one electrically conductive electrode such that, with the menstrual collector inserted into the vaginal canal, at least a portion of the cord is arranged to be located exterior to the vaginal canal.

11. The intravaginal electrical stimulation device of claim 10, further comprising a battery connected to the cord such that, with the menstrual collector inserted into the vaginal canal, the battery is arranged to be located exterior to the vaginal canal.

12. The intravaginal electrical stimulation device of claim 1, wherein the at least one pelvic nerve is a hypogastric nerve, a pudendal nerve, or a combination of the same.

13. The intravaginal electrical stimulation device of claim 1, wherein the pelvic condition is primary dysmenorrhea or secondary dysmenorrhea.

14. An intravaginal electrical stimulation device comprising
a menstrual collector configured to be inserted into a vaginal canal of a user, the menstrual collector including a body shaped to define an interior region that provides a fluid-collection reservoir and a rim coupled to the body and formed to include an opening into the interior region, and
at least one electrically conductive electrode coupled to the menstrual collector such that, with the menstrual collector inserted into the vaginal canal, at least a portion of the at least one electrically conductive electrode is arranged to contact a vaginal wall of the vaginal canal, the at least one electrically conductive electrode configured to electrically stimulate at least one pelvic nerve through the vaginal wall so that discomfort or pain associated with a pelvic condition or a procedure is alleviated.

15. The intravaginal electrical stimulation device of claim 14, wherein the rim of the menstrual collector includes an anterior portion and a posterior portion opposite the anterior portion, and wherein the anterior portion of the rim is a first height from a bottom end of the body and the posterior portion of the rim is a second height from the bottom end of the body, the second height being greater than the first height to conform to a shape of the vaginal canal.

16. The intravaginal electrical stimulation device of claim 14, wherein the at least one electrically conductive electrode includes a first electrode coupled to the rim of the menstrual collector and a second electrode coupled to a posterior side of the body of the menstrual collector that is arranged to contact the vaginal wall, and wherein the vaginal wall is a posterior wall of the vaginal canal.

17. The intravaginal electrical stimulation device of claim 14, wherein the at least one electrically conductive electrode includes a plurality of electrically conductive electrodes coupled to a posterior side of the body of the menstrual collector that is arranged to contact the vaginal wall, and wherein the vaginal wall is a posterior wall of the vaginal canal.

18. A method of intravaginal electrical stimulation comprising
inserting a menstrual collector into a vaginal canal to locate at least one electrically conductive electrode coupled to the menstrual collector in the vaginal canal,
contacting a vaginal wall of the vaginal canal with at least a portion of the at least one electrically conductive electrode, and
electrically stimulating at least one pelvic nerve through the vaginal wall via the at least one electrically conductive electrode thereby reducing discomfort or pain associated with a pelvic condition or a procedure.

19. The method of claim 18, wherein the menstrual collector includes a body shaped to define an interior region that provides a fluid-collection reservoir and a rim coupled to the body and formed to include an opening into the interior region, wherein the rim includes an anterior portion and a posterior portion opposite the anterior portion, the anterior portion of the rim is a first height from a bottom end of the body and the posterior portion of the rim is a second height from the bottom end of the body that is greater than the first height.

20. The method of claim 19, wherein the step of inserting includes inserting the menstrual collector into the vaginal canal so that the posterior portion of the rim engages a cervix of the user.
